(19) United States
(12) Patent Application Publication
(10) Pub. No.: US 2026/0274472 A1
(43) Pub. Date: Sep. 17, 2026

(54) METHOD OF PRODUCTION

(71) Applicant: Novartis AG, Basel (CH)

(72) Inventor: Geert Vandenbossche, Destelbergen (BE)

(21) Appl. No.: 19/480,099

(22) PCT Filed: May 8, 2024
(86) PCT No.: PCT/IB2024/054472

§ 371 Date: Oct. 30, 2025

Related U.S. Application Data
(60) Provisional application No. 63/500,937, filed on May 9, 2023.

(51) Int. Cl. B65B 3/00; A61K 45/06; B65B 55/02; B65B 57/00; B65B 65/00

(52) U.S. Cl. CPC B65B 3/006; A61K 45/06; B65B 55/027; B65B 57/005; B65B 65/00; B65B 2220/16

(57) ABSTRACT

The present disclosure relates to a method of producing a packaged radiopharmaceutical drug product by operating the different manufacturing steps including inspection in various cold and shielded units of cleanliness zoning requirements of Grade A, B, C, or CNC. It further relates to the manufacturing units comprising technical equipment required for said method, the drug product obtained by said method, as well as its technical and medical use.

### FIELD OF THE INVENTION

The present disclosure relates to a method of producing a packaged radiopharmaceutical drug product by operating the different manufacturing steps including inspection in various cold and shielded units of cleanliness zoning requirements of Grade A, B, C, or CNC. It further relates to the manufacturing units comprising technical equipment for said method, the drug product obtained by said method, as well as its technical and medical use.

### BACKGROUND OF THE INVENTION

The manufacturing of radiopharmaceutical drug products is challenging. Those products are typically administered parenterally and therefore require aspetic manufacturing in a strict quality controlled environment to ensure sterility of the final product.

Due to the radioactivity of said products the manufacturing is to be done in shielded units. The current practice is to perform the entire production of a radiopharmaceutical product in a single shielded isolator that allows manipulations via gloves or gripping devices from outside the isolator and material locks for educt and product transfers into and out of said isolator. Said isolator has to meet cleanliness zoning requirement Grade A for an aseptic production of a sterile radiopharmaceutical product.

Every process material that is or was inside said box is potentially contaminated by radioactivity and thus require particular waste management.

Further, the quality controls and inspection of the final radiopharmaceutical product is also challenging. A container closure integrity test has to be done but bears the risk to contaminate the entire production unit in case of a identified leakage. Also, the visual inspection for particulate matter by naked eye of the operator from a short distance is difficult due to the radioactivity of the product which can only be visually monitored through thick lead glasses of imperfect transparency.

During the past years, several new radiopharmaceutical drug products have been demonstrated by pivotal clinical trials to be a safe and effective cancer treatment option (e.g. LUTATHERA and PLUVICTO). With the approvals of those drugs and the their broader clinical use, the demand for such radiopharmaceutical products has significantly increased and require production on large scale. Therefore, there is a high need for a more economic production on industrial scale.

### SUMMARY OF THE INVENTION

The present disclosure provides such a highly economic production method on an unprecedented industrial scale and is suitable for implementation as automated process line. It reduces significantly radioactive contaminated waste and de-risks radioactive contamination of the process line during the manufacturing process and product quality control steps and provides highly efficient and reliable product inspection solutions. It further provides a primary and secondary packaging solutions that are novel for radiopharmaceutical products.

The present disclosure provides said production method in the following aspects:

A method for producing a packaged radiopharmaceutical drug product (e.g. a radioligand drug product) comprising the steps of:
(1) Synthesizing and compounding a radiopharmaceutical drug substance solution in one or more shielded units of (cleanliness zoning requirement) Grade C to obtain a radiopharmaceutical drug product;
(2) Providing primary packaging materials, e.g. by (outer-layer-)unpacking in one or more cold units of Grade C, sanitizing in one or more cold units of Grade B, and (inner-layer-)unpacking said packaging materials in one or more cold units of Grade A;
(3) Filling the radiopharmaceutical drug substance solution obtained by step (1) into the primary packaging materials as provided and unpacked by step (2) in a shielded unit of Grade A to obtain the primarily packaged radiopharmaceutical drug product;
(4) Inspecting the primarily packaged radiopharmaceutical drug product obtained by step (3) in one or more shielded units of Grade “Controlled, non-classified” (CNC); and
(5) optionally, placing the primarily packaged radiopharmaceutical drug product as inspected by step (4) into a shielded secondary packaging material in one or more shielded units of Grade CNC to obtained a primarily and secondarily packaged radiopharmaceutical drug product.

As described above, to prevent radio-active waste, all packaging components are unpacked in a cold area before entering in the filling unit. This prevents contamination risk. All tubs and bags can therefore be treated as non-radioactive and destroyed in a cheaper way.

The method for producing a packaged radiopharmaceutical drug product as described above, wherein in step (4) a container closure integrity test (CCIT) of the primarily packaged radiopharmaceutical drug product is performed as one of the first inspecting steps, preferably, the CCIT is performed with the primarily packaged radiopharmaceutical drug product present in a(n additional) container that allows capturing/segregation of the drug product in said container in case of any leakage detection or risk of spillage.

As a unique feature, the CCIT (container closure integrity testing) is done as a first step after filling. In this way,
a) the vial is kept in a container to collect any liquid spilled;
b) the vial is tested on integrity;
c) in case of leakage, the cup and vial are segregated and do not contaminate the line
This setup serves 2 goals:
Beside of ensuring that the container is integer (avoiding contamination risk for user and patient), this also ensures that a leaking container would not contaminate the production line.

The method for producing a packaged radiopharmaceutical drug product as described above, wherein in step (4) a camera assisted visual inspection (VI) is performed in the form of video recordation, preferably, said video recordation is then evaluated by naked-eye.

Visual inspection is typically done either:
a) by naked eye looking at the vial, but this is not possible for radiopharmaceutical products because of the radioactivity and low resolution behind leaded glass; or
b) by a camera and image analysis system is used to detect particles automatically (image analysis)
The novel approach of the present disclosure is to record a video and evaluate said recording with the naked eye.

The method for producing a packaged radiopharmaceutical drug product as described above, wherein in step (1) the synthesizing step is performed several times, preferable up to 4 times, preferably said synthesizing step is performed in parallel in several synthesizing units, and the resulting synthesized radiopharmaceutical drug substance solutions are then collected as one combined (bulk) volume for compounding.

The method for producing a packaged radiopharmaceutical drug product as described above, wherein the primary packaging material comprises a glass vial and an all-plastic push-fit cap, wherein the rubber stopper is pre-assembled in the cap, for example, a RayDyLyo cap by ARaymond.

The method for producing a packaged radiopharmaceutical drug product as described above, wherein the shielded secondary packaging material comprises a lead pot with a corresponding lid, preferably with a tamper evident application, preferably said lead pot and its lid is free of a paint-coating, preferably said lead pot and its lid are plastic-coated, preferably a rubber ring is present between pot and lid for leakage-safe closure.

The method for producing a packaged radiopharmaceutical drug product as described above, wherein the drug substance carrying containers and tubings connecting said containers are flushed with protective gas/inert gas, for example nitrogen (N₂) and/or argon (Ar).

The method for producing a packaged radiopharmaceutical drug product as described above, wherein the solutions involved in the method are transferred from one container to the other via tubings by applying overpressure of protective gas/inert gas, for example nitrogen (N₂) and/or argon (Ar), preferably, said transfer is preceded by testing the tubings for flow and leakage, e.g. by a pressure test with said protective gas/inert gas.

The method for producing a packaged radiopharmaceutical drug product as described above, wherein said product is produced on a scale of at least 30 Ci, preferably at least 32 Ci, more preferably 35 Ci, even more preferably at least 40 Ci.

### BRIEF DESCRIPTION OF THE DRAWINGS

FIG. 1 shows a top view of a linearly arranged example production line (with two synthesis units and two compounding units). Shielded unit for beta (optional alpha)/gamma irradiation; Thin line: Cold unit; Dash line: Optional.

FIG. 2 shows a schematic overview of the modules of an example production line (with 4 synthesis units and one compounding unit). Thick line: Shielded unit for beta (optional alpha)/gamma irradiation; Thin line: Cold unit; Dash line: Optional. “BC” indicates the Grade A Filling unit outlet of first Grade B and then Grade C.

### DETAILED DESCRIPTION OF THE INVENTION

Herein after, the present disclosure is described in further detail and is exemplified.

### EMBODIMENTS

The methods of treatments of the present disclosure are provided in particular as following embodiments:

1. A method for producing a packaged radiopharmaceutical drug product (e.g. a radioligand drug product) comprising the steps of:
(1) Synthesizing and compounding a radiopharmaceutical drug substance solution in one or more shielded units of (cleanliness zoning requirement) Grade C to obtain a radiopharmaceutical drug product;
(2) Providing primary packaging materials (e.g. as ready-to-use materials, in sealed tubs), e.g. by (outer-layer-)unpacking in one or more cold units of Grade C, sanitizing in one or more cold units of Grade B, and (inner-layer-)unpacking (e.g. removing the Tyvek sheet from the tub) said packaging materials in one or more cold units of Grade A;
(3) Filling the radiopharmaceutical drug substance solution obtained by step (1) into the primary packaging materials as provided and unpacked by step (2) in a shielded unit of Grade A to obtain the primarily packaged radiopharmaceutical drug product;
(4) Inspecting the primarily packaged radiopharmaceutical drug product obtained by step (3) in one or more shielded units of Grade “Controlled, non-classified” (CNC); and
(5) optionally, placing the primarily packaged radiopharmaceutical drug product as inspected by step (4) into a shielded secondary packaging material in one or more shielded units of Grade CNC to obtained a primarily and secondarily packaged radiopharmaceutical drug product.

In certain embodiments, the unpacking and sanitization in step (2) is done in the following way:
In a cold unit of Grade C, the outer bags surrounding a sealed tubs are removed.
In a cold unit of Grade B, sanitization of the sealed tubs is done.
In a cold unit of Grade A, unsealing of the tubs is done, e.g. by removing the Tyvek sheet from the tub.

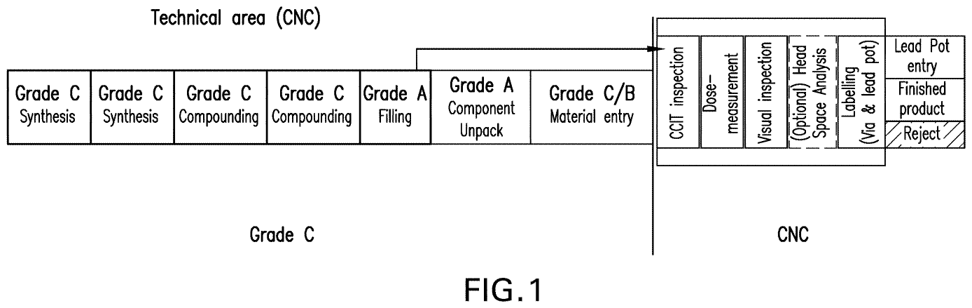

In certain embodiments, the units are linearly arranged, e.g. as shown in FIG. 1. The linearly arranged units are considered as preferred option.

2. The method for producing a packaged radiopharmaceutical drug product according to embodiment 1, wherein in step (4) a container closure integrity test (CCIT) of the primarily packaged radiopharmaceutical drug product is performed as one of the first inspecting steps, preferably, the CCIT is performed with the primarily packaged radiopharmaceutical drug product present in a(n additional) container that allows capturing/segregation of the drug product in said container in case of any leakage detection or risk of spillage.

3. The method for producing a packaged radiopharmaceutical drug product according to embodiment 1 or 2, wherein in step (4) a camera assisted visual inspection (VI) is performed in the form of video recordation, preferably, said video recordation is then evaluated by naked-eye.

4. The method for producing a packaged radiopharmaceutical drug product according any one of the preceding embodiments, wherein in step (1) the synthesizing step is performed several times, preferable up to 4 times, preferably said synthesizing step is performed in parallel in several synthesizing units, and the resulting synthesized radiopharmaceutical drug substance solutions are then collected as one combined (bulk) volume for compounding.

5. The method for producing a packaged radiopharmaceutical drug product according any one of the preceding embodiments, wherein in step (1) the synthesizing step is performed in a cassette-based synthesis units, preferably said cassette-system manufacturing synthesizer unit as provided e.g. by Trasis, preferably MiniAlO as described e.g. in WO 2020/089379 A1, preferably, two double synthesis units by Trasis are used.

6. The method for producing a packaged radiopharmaceutical drug product according any one of the preceding embodiments, wherein in step (1) the compounding step comprises the sub-steps of:
(i) Filling the synthesized solution(s) into a container, e.g. a (bulk) vial or bottle to obtain a (concentrated) mother solution;
(ii) optionally, measuring the radioactivity of said (concentrated) mother solution;
(iii) optionally, transferring said (concentrated) mother solution into another container, preferably a flexible plastic bag,
(iv) adding a dilution solution in an amount to obtain the drug substance solution in the desired radioactive concentration; and
(v) mixing the solution as result of step (iv), preferably and if applicable, by moving the flexible plastic bag appropriately, to obtain a homogeneous diluted drug substance solution for filling.

7. The method for producing a packaged radiopharmaceutical drug product according any one of the preceding embodiments, wherein in step (2) the packaged primary packaging material is (bio)decontaminated/sanitized, preferably with vaporized hydrogen peroxide (VHP), before being transferred from the unit of Grade B to the unit of Grade A.

8. The method for producing a packaged radiopharmaceutical drug product according any one of the preceding embodiments, wherein in step (4) inspecting comprises the following sub-steps:
(i) CCIT;
(ii) radioactive dose measurement/calibration;
(iii) visual inspection; and
(iv) optionally, head space analysis;
preferably in said order.

9. The method for producing a packaged radiopharmaceutical drug product according any one of the preceding embodiments, wherein the primary packaging material comprises a glass vial and an all-plastic push-fit cap, wherein the rubber stopper is pre-assembled in the cap, for example, a RayDyLyo cap by ARaymond.

10. The method for producing a packaged radiopharmaceutical drug product according any one of the preceding embodiments, wherein the shielded secondary packaging material comprises a lead pot with a corresponding lid, preferably with a tamper evident application, preferably said lead pot and its lid is free of a paint-coating, preferably said lead pot and its lid are plastic-coated, preferably a rubber ring is present between pot and lid for leakage-safe closure.

11. The method for producing a packaged radiopharmaceutical drug product according any one of the preceding embodiments, wherein the drug substance carrying containers and tubings connecting said containers are flushed with protective gas/inert gas, for example nitrogen (N₂) and/or argon (Ar).

12. The method for producing a packaged radiopharmaceutical drug product according any one of the preceding embodiments, wherein the solutions involved in the method are transferred from one container to the other via tubings by applying overpressure of protective gas/inert gas, for example nitrogen (N₂) and/or argon (Ar), preferably, said transfer is preceded by testing the tubings for flow and leakage, e.g. by a pressure test with said protective gas/inert gas.

13. The method for producing a packaged radiopharmaceutical drug product according any one of the preceding embodiments, wherein the solutions used in the method are purged and/or flushed with protective gas/inert gas, for example nitrogen (N₂) and/or argon (Ar).

14. The method for producing a packaged radiopharmaceutical drug product according any one of the preceding embodiments, wherein the at least the steps (1)-(3) are automated, preferably also the steps (4) and (5) are automated, with the exception of the naked-eye evaluation of the video recordation in step (4).

15. The method for producing a packaged radiopharmaceutical drug product according any one of the preceding embodiments, wherein said product is produced on a scale of at least 30 Ci, preferably at least 32 Ci, more preferably 35 Ci, even more preferably at least 40 Ci.

16. A packaged radiopharmaceutical drug product obtained (and/or obtainable) by the methods as described in anyone of the preceding embodiments.

17. A manufacturing unit (e.g. in the form of a(n automated) process line) comprising the technical equipment and facilities for producing a packaged radiopharmaceutical drug product according to the methods as described in anyone of the preceding embodiments.

18. Use of the manufacturing unit according to embodiment 17 for producing the packaged radiopharmaceutical drug product.

19. A method of treating a cancer comprising administering to a patient in need thereof a therapeutically effective amount of the radiopharmaceutical drug product contained in the packaged radiopharmaceutical drug product according to embodiment 16.

20. The radiopharmaceutical drug product contained in the packaged radiopharmaceutical drug product according to embodiment 16 for use in the treatment of a cancer.

21. Use of the radiopharmaceutical drug product contained in the packaged radiopharmaceutical drug product according to embodiment 16 for the preparation of a medicament for the treatment of a cancer.

### Definitions (and Further Embodiments and Embodiment Feature Descriptions)

“about” in respect of a value means±25%, preferably ±20%, more preferably ±15%, even more preferably ±10%, even more preferably ±5%.

### Radiopharmaceutical Drug Product:

In certain embodiments of the present disclosure, the term radiopharmaceutical drug product may refer to any drug product that comprises a radioactive material, e.g. a radioactive nuclide, radionuclide or radioisotope, e.g. F-18, Cu-64, Cu-67, Ga-68, Y-90, Tc-99m, I-131, Tb-161, Er-169, Lu-177, Pb-212, Bi-212, Ra-223, Ac-225. The radionuclide may be present in such drug product in the form of a salt, e.g. as aqueous solution (e.g. Radium chloride solution, Xofigo), or covalently bound to an organic molecule, e.g. 18F-DCFPyL, Pylarify), or complexed in a chelator, whereby said chelator might be conjugated (e.g. via a linker) to a target-binding moiety, i.e. a radioligand, e.g. 177Lu-DOTATATE, Lutathera, or 177Lu-PSMA-617, Pluvicto.

### Radioligand Drug Product:

In certain embodiments of the present disclosure, the term radioligand drug product may refer to a drug product comprising a radioligand for therapeutic use (i.e. a radioligand therapeutic (RLT) agent or for imaging use (i.e. a radio ligand imaging (RLI) agent).

The RLT agent may comprise at least two components:
(1) a radionuclide component; and
(2) a ligand component;
wherein said radionuclide component (1) comprises:
(a) at least one radionuclide, preferably selected from the group consisting of alpha particle-emitting radionuclide, beta-minus electron-emitting radionuclide and Auger electron-emitting radionuclide, more preferably a beta-minus electron-emitting radionuclide; and
wherein said ligand component (2) comprises:
(b) at least one target binding moiety (e.g. a PSMA-binding moiety, or an somatostatin receptor targeting moiety);
(c) optionally at least one chelator for chelating the radionuclide or a salt comprising the radionuclide, or a prosthetic group residue from a radiohalogenation reaction;
(d) optionally at least one linker connecting the PSMA-binding moiety (b) with the chelator or prosthetic group component (c), preferably said linker is a chemical moiety or a covalent bond;
(e) optionally at least one additional moiety that alters the systemic circulation time, tumor uptake, and/or biodistribution of the RLT agent, preferably said altering moiety comprises oxyethylene units, e.g. an oligo- or polyoxyethylene —(—CH2-CH2-O—)n- with n=2-100, or is an albumin-binding moiety (e.g. Evans blue, 4-(p-iodophenyl)butyric acid, 4-(p-methylphenyl)butyric acid, ibuprofen).

The radionuclide may be selected from the group consisting of Lu-177, Tb-161, 1-131, Tc-99m, Y-90, Sc-47, Cu-67, Re-188, Pb-212, Bi-213, Ac-255, and Th-227, preferably selected from the group consisting of Lu-177 and Tb-161.

The radionuclide may be a beta-minus electron emitting radionuclide with an half-life of from about 2 to about 10 days, preferably from about 5 to about 10 days, more preferably from about 6 to about 8 days, even more preferably about 6 or about 7 days; and a beta-minus electron maximum energy of from about 0.3 to about 1.0 MeV, preferably from about 0.5 to about 0.8 MeV, more preferably about 0.5, 0.6, 0.7 or 0.8 MeV, even more preferably about 0.5 or about 0.6 MeV.

The radionuclide may be a beta-minus electron emitting radionuclide with an absorbed electron energy fraction per decay of from 100 to 300 keV/decay, from 120 to 250 keV/decay, from about 150 keV/decay (e.g. for Lu-177: 147 keV) to about 200 keV/decay (e.g. for Tb-161: 196 keV/decay).

The ligand may be selected from the group consisting of PSMA-617, PSMA I&T, PSMA-R2, MIP-1095, MIP-1545, MIP-1555, MIP-1557, MIP-1558, CTT1403, FC705, BAY-2315497, TLX592, PSMA-TCC, rhPSMA, rhPSMA-7, rhPSMA-7.3, rhPSMA-10.1, Ludotadipep, PNT2001, PNT2002, PSMA-7 I&T, EB-PSMA-617, PSMA-ALB-02, PSMA-ALB-053, PSMA-ALB-056, P16-093, PSMA-93, and RPS-074 (or any albumin-binder-modified versions thereof, e.g. Evans blue (EB)-PSMA-617), preferably selected from the groupd consisting of PSMA-617, PSMA I&T, and PSMA-R2.

The PSMA-binding moiety may comprise at least two amino acids connected via an urea or phosphoramide group, preferably glutamate-urea-lysine (GUL), or an antibody or fragment thereof, e.g. TLX591, J591, rosopatamab, IAB2M, GCP-05, 1H8H5, SP29, or FOLHI.

The radioligand therapeutic agent may be selected from the group consisting of [177Lu]Lu-PSMA-617 (lutetium (177Lu) vipivotide tetraxetan), [177Lu]Lu-EB-PSMA-617 (Evans Blue modified [177Lu]Lu-PSMA-617), and [177Lu]Lu-PSMA I&T (lutetium (177Lu) zadavotide guraxetan), [161Tb]Tb-PSMA-617 (terbium (161Tb) vipivotide tetraxetan), [161Tb]Tb-EB-PSMA-617 (Evans Blue modified [161Tb]Tb-PSMA-617), and [161Tb]Tb-PSMA I&T (terbium (161Tb) zadavotide guraxetan), preferably [177Lu]Lu-PSMA-617 (lutetium (177Lu) vipivotide tetraxetan) or [161Tb]Tb-PSMA-617 (terbium (161Tb) vipivotide tetraxetan), more preferably [177Lu]Lu-PSMA-617 (lutetium (177Lu) vipivotide tetraxetan).

The PSMA-binding moiety may be glutamate-urea-lysine (GUL) and the linker may comprise the residues of (2-naphthyl)-L-alanine and trans-4-aminomethyl-cyclohexanecarboxylic acid or the linker comprises the residues of an optionally substituted phenyl-alanine and/or optionally substituted tyrosine, preferably, a phenyl-alanine and a substituted tyrosine, more preferably a phenyl-alanine and a iodo-substituted tyrosine, even more preferably a D-phenyl-alanine and a iodo-substituted D-tyrosine.

The ligand may be selected from the group consisting of DOTA-OC: [DOTA0,D-Phe1]octreotide, DOTA-TOC: [DOTA0,D-Phe1,Tyr3]octreotide, edotreotide (INN), DOTA-NOC: [DOTA0, D-Phe1,1-Nal3]octreotide, DOTA-TATE: [DOTA0,D-Phe1,Tyr3]octreotate, DOTA-Tyr3-Octreotate, DOTA-d-Phe-Cys-Tyr-d-Trp-Lys-Thr-Cys-Thr (cyclo 2,7), oxodotreotide (INN), DOTA-LAN: [DOTA0,D-β-Na1]lanreotide, DOTA-VAP: [DOTA0,D-Phe1,Tyr3]vapreotide, Satoreotide trizoxetan, and Satoreotide tetraxetan.

The radioligand imaging agent may comprise:
(1) a radionuclide component; and
(2) a ligand component;
wherein said radionuclide component (1) comprises:
(a) at least one positron-emitting radionuclide; and
wherein said ligand component (2) comprises:
(b) at least one target-binding moiety (e.g. a PSMA-binding moiety, or an somatostatin receptor binding peptide, e.g. oxodotreotide or edotreotide);
(c) optionally at least one chelator for chelating the radionuclide or a salt comprising the radionuclide, or a prosthetic group residue from a radiohalogenation reaction;
(d) optionally at least one linker connecting the PSMA binding moiety (b) with the chelator/prosthetic group component (c), preferably said linker is a chemical moiety or a covalent bond.

The radionuclide for the RLI agent may be selected from the group consisting of F-18, Ga-67, Ga-68, and Cu-64.

The ligand for the RLI agent may be selected from the group consisting of PSMA-11 (gozetotide), DCPyL (if labeled with 18F available as PYLARIFY, INN: piflufolastat F-18, also referred to shortly as PyL), MIP-1404, rhPSMA 07, PSMA-1007, THP-PSMA, iPSMA, P16-093, PSMA-93, rhPSMA, rhPSMA-7, rhPSMA-7.3, PSMA-7, and PSMA I&T.

The ligand may be selected from the group consisting of DOTA-OC: [DOTA0,D-Phe1]octreotide, DOTA-TOC: [DOTA0,D-Phe1,Tyr3]octreotide, edotreotide (INN), DOTA-NOC: [DOTA0, D-Phe1,1-Nal3]octreotide, DOTA-TATE: [DOTA0,D-Phe1,Tyr3]octreotate, DOTA-Tyr3-Octreotate, DOTA-d-Phe-Cys-Tyr-d-Trp-Lys-Thr-Cys-Thr (cyclo 2,7), oxodotreotide (INN), DOTA-LAN: [DOTA0,D-β-Nal1]lanreotide, DOTA-VAP: [DOTA0,D-Phe1,Tyr3]vapreotide, Satoreotide trizoxetan, and Satoreotide tetraxetan.

### Synthesizing (Step 1):

In certain embodiments of the present disclosure, the term “synthesizing” may refer to radiolabeling a target-binding ligand, e.g. by complexing a radiometal into the chelator of the ligand. Said radiolabeling may require heating a solution of the radionuclide and the ligand for a certain time (e.g. about 5-15 min) and at a certain temperature (e.g. about 95° C.). The solution may contain a buffer for ensuring the pH is optimal for the complex formation (e.g. e.g. an acetate buffer for a pH of about 4-6), a stabilizer against radiolytic degradation (autoradiolysis), e.g. gentisic acid or ascorbic acid, or salts thereof. The result of this radiolabling may be referred to as mother solution or concentrated mother solution.

The synthesizing step may be performed in a cassette-based synthesis units, preferably said cassette-system manufacturing synthesizer unit as provided e.g. by Trasis, preferably MiniAIO as described e.g. in WO 2020/089379 A1 (the content of it are incorperated by reference), preferably, two double synthesis units by Trasis are used.

### Compounding (Step 1):

In certain embodiments of the present disclosure, the term “compounding” may refer to diluting the concentrated mother solution, e.g. by adding water or an aqueous solution optionally comprising a sequestering agent (e.g. DTPA), a stabilizer against radiolytic degradation (autoradiolysis), e.g. gentisic acid or ascorbic acids, or salts thereof, optionally also a isotonic agent, e.g. NaCl, to adjust the tonicity of the resulting solution, and optionally a pH adjusting agent, e.g. NaOH or HCl to adjust the pH to bring the pH closer to physiological pH. The aim of this dilution step is to adjust the radioactivity concentration to a target concentration that then suitable for the filling step to produce the patient dose units, e.g. about 1000 MBq/mL (27 mCi/mL).

The compounding step may comprise the sub-steps of:
(i) Filling the synthesized solution(s) into a container, e.g. a (bulk) vial or bottle to obtain a (concentrated) mother solution;
(ii) optionally, measuring the radioactivity of said (concentrated) mother solution;
(iii) optionally, transferring said (concentrated) mother solution into another container, preferably a flexible plastic bag,
(iv) adding a dilution solution in an amount to obtain the drug substance solution in the desired radioactive concentration; and
(v) mixing the solution as result of step (iv), preferably and if applicable, by moving the flexible plastic bag appropriately, to obtain a homogeneous diluted drug substance solution for filling.

### Shielded Unit:

In certain embodiments of the present disclosure, the term “shielded unit” may refer to a manufacturing unit that is shielded by a material suitable to prevent that ionizing radiation (e.g. alpha-particle, beta-minus electrons, gamma-rays) is leaving said unit. Therefore, within said shielded units radioactive material can be safely handled.

### Cold Unit:

In certain embodiments of the present disclosure, the term “cold unit” may refer to those manufacturing units that are not shielded against ionizing radiation. Therefore, in those cold units, no radioactive material should be handled.

### Primary Packaging Material:

In certain embodiments of the present disclosure, the term “primary packaging material” may refer to those containers that are in direct contact with the drug product, e.g. with the radioactive solution. Such primary packaging material may be a glass vial with a rubber stopper and a cap; or a glass vial with an all-plastic push-fit cap, wherein the rubber stopper is pre-assembled in the cap, for example, a RayDyLyo cap by Araymond; or a cartridge, e.g. a glass cylinder that is closed by rubber pieces, wherein at least one rubber piece is movable, said cartridge may be then used by loading into syringe devices; or a prefilled syringe. Such primary packaging material may be provided in tubs that are sealed, e.g. by foils, such as those commercialized under the name Tyvek by DuPont (e.g. made out of high-density polyethylene (HDPE)). Such sealed tubs containing the primary packaging material may be then wrapped in a further bag.

### Inspecting (Step 4):

In certain embodiments of the present disclosure, the term “inspecting” may refer to steps that control the quality and integrity of the drug product. Those inspecting or inspection steps may be in-process controls or final product controls. Those inspection steps may be container closure integrity tests (CCIT), radioactive dose measurement/calibration, visual inspection, head space analysis, or any other product quality controls.

### Cleanliness Zoning Requirements:

Clean room and clean air devices are classified in accordance with EN ISO 14644-1. The maximum permitted airborne particle concentration for each grade is given in the following table.

| | Maximum permitted number of particles per | | | |
|---|---|---|---|---|
| | m3 equal to or greater than the tabulated size | | | |
| | At Rest | | In Operation | |
| Grade | 0.5 μm | 5.0 μm | 0.5 μm | 5.0 μm |
| A | 3 520 | 20 | 3 520 | 20 |
| B | 3 520 | 29 | 352 000 | 2 900 |
| C | 352 000 | 2 900 | 3 520 000 | 29 000 |
| D | 3 520 000 | 29 000 | Not Defined | Not Defined |

The approximate airborne particle classification per ISO 14644-1 standards is listed in the table below.

| Approximate airborne particle | |
|---|---|
| classification per ISO 14644-1 standards | |
| At Rest | In Operation |

| Grade | 0.5 μm | 5.0 μm | 0.5 μm | 5.0 μm |
|---|---|---|---|---|
| A | ISO 5 | ISO 5 | ISO 5 | ISO 5 |
| B | ISO 5 | ISO 5 | ISO 7 | ISO 7 |
| C | ISO 7 | ISO 7 | ISO 8 | ISO 8 |
| D | ISO 8 | ISO 8 | Not Defined | Not Defined |

The Grade “Controlled, Non-Classified” (CNC) refers to a cGMP manufacturing area designed to produce a consistent and controlled environment, but not necessarily monitored to a given environmental classification. Non-classified areas may have uncontrolled airborne particle amounts, but temperature and humidity are still maintained. Non-classified area may be a non-process area or area in which the product does not comes in direct contact with air.

### REFERENCES

All publications, patents and patent applications referred to herein are incorporated by reference in their entirety to the same extent as if each individual publication, patent or patent application was specifically and individually indicated to be incorporated by reference in its entirety.

### EXAMPLES

Hereinafter, the present invention is described in more details and specifically with reference to the examples, which however are not intended to limit the present invention.

### Example 1

### General Information, Preparations

The production of radiopharmaceuticals occurs in the synthesis modules and is carried out every time with a new cassette, new set of reagents and new tubing. All consumables are sterile and single use.

All the transfer lines of the solution are sterile and single use. In every step of the production and distribution processes, the starting materials, the intermediate and final products are in contact with sterile, single-use material.

The manufacturing isolator is decontaminated prior to each manufactured batch using qualified vaporized hydrogen peroxide cycles. The isolator cannot be cleaned immediately after production due to the high exposure of radioactivity and should be done when radioactivity levels have dropped or when all items have been consolidated and can be removed in a way that promotes ALARA practices.

The cleaning of the hot cells is therefore carried out before each daily production cycle. The clean room is cleaned every day after the production according to the procedure.

The sterile radiopharmaceuticals for injection are produced by utilizing automated synthesis module and diluted under aseptic conditions and using aseptic techniques. The radionuclide used is supplied by IDB, MURR, or ITM.

The synthesis is performed using a radiosynthesis module. This module is contained within a shielded hot cell and utilizes a 0.2 um filtration as the drug substance is transferred into the hot cell. The module is automated and controlled by a software which includes the check and registration of parameters of the method. The software also records the conditions under which manufacturing occurs and reports these conditions.

The aqueous solution containing ¹⁷⁷LuCl₃ is transferred into the radio synthesis module by capillary tubes into a reactor vessel. The Reaction Buffer and ligand peptide are transferred into the same reactor vessel. The solution goes through the radio-labelling step for about five minutes prior to being transferred to the dispensing hot cell.

The drug substance is filtered through a 0.2 um filter and is transferred into a sterile mother vial. The drug substance is then diluted to the drug product and dispensed through sterilizing filter into a sterile container.

### Packaging and Labeling

The packaging of radiopharmaceuticals requires specific measures of radioprotection. The packaging is essentially composed of three parts: the glass vial (primary packaging), the lead container (sub-secondary packaging) and type A container (secondary packaging).

The packaging system follows ADR regulations relating to the transport of hazardous goods by road and is temperature controlled using gel packs to ensure the stability of the product.

The labelling is done on primary packaging, sub-secondary packaging and secondary packaging.

### Storage of Finished Products

Because of the short shelf life (72 hours to 120 hours), radiopharmaceuticals are shipped to hospitals after lot release by Quality. In general, doses are produced on day one, released by Quality and shipped to hospitals on day two, to then be administrated to patients on day two or three following manufacturing completion.

### Automated Process Line

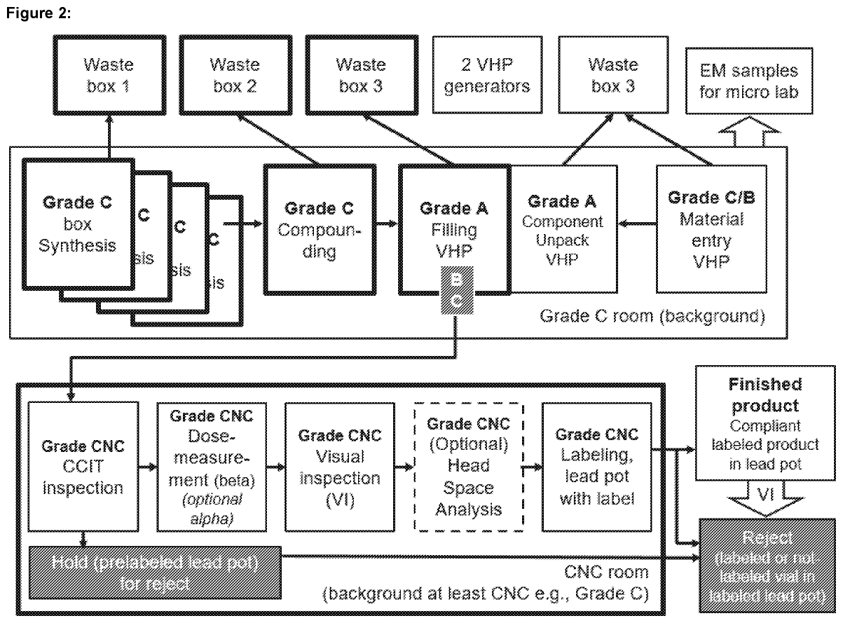

Schematic overview of modules within new filling line is provided by FIG. 1 for an overview and FIG. 2 and FIG. 3 for the details.

The units of the process line are described in the following.

Grade C unit (Synthesis)

### Description of Activity:

Activity is measured upfront
Materials added via open door (including transfer and connection of radio-isotope)
Synthesis
Optional: automatic system to install all needles into reagent containers-including radio-isotope
Transfer of product via disposable tubing to compounding cell (note: tube installed prior to startup of the campaign)
Transfer of waste to waste box 1
EM sampling (start)

### Equipment:

Isolatortype box grade C/shielded
Optional: Tool to install/verify reagent
N₂ for transfers
Transfer system to compounding
Transfer system to waste box1
Gloves in plexipanel & access ports in shielded door
2 (double) synthesis units (Trasis) & disposables are installed by operator

### Critical Aspects:

Grade C conditions at rest and during operations
IQ/OQ provided by the equipment supplier
Qualification of the grade with running equipment, synthesis equipment and process (PQ) is done by done by operator

### Waste Box

### Description of Activity:

Collection of waste from synthesis boxes and filling unit respectively
Transfer mechanism and lay-out to be defined by the supplier
Proposal for maximal compression of waste volume to be made by the supplier

### Equipment:

If applicable: equipment to reduce volume of waste
Boxes can be in non-classified environment (shielded)
Gloves in plexipanel & access ports in shielded door

### Critical Aspects:

Non GMP critical activity

### Grade C Compounding

### Description of Activity:

Materials are added via open door. Including sterile, preassembled disposables
Area is used for dose calibration of radioisotope for synthesis or concentrated mother solution
Product specific: optionally flush closed system with N₂ at all relevant steps
Able to manage following sequence:
   1. Addition & mixing of compounding buffer to saline*
   2. Labelled product from synthesis cell is transferred in via disposable tubing.
   3. Dose measurement of the mother solution (250 ml)
   4. Addition of mother solution to buffer solution
   5. Adjustment & mixing to final concentration
Backup scenario needs to be developed in case (one) of the synthesis fails (software can can compensate for dilution factor in case of partial reject)
Batch size from 0.5 to 5 liter solution.
Finished product is transferred via disposable tubing
   1. Note: the solution remains in the compounding unit during filling.
   2. Note: several tubes installed prior to startup of the campaign)
EM sampling (at start)
   * This activity must be done separated in time or space:
Or the buffer is prepared, mixed, flushed with nitrogen outside the shielded units
Or the buffer is prepared, mixed, flushed with nitrogen outside in the shielded units before any radioactive material is added.
Note: the first solution requires an extra balance, rocking table and pump.

### Equipment:

Isolatorbox grade C/Shielded
Sterility grade disposable filter
Balance
Peristaltic pump and/or nitrogen for transfers
Dose calibrator (range: incoming Lu/labelled API)
Mixing system to be proposed by supplier (tilting table, recirculation)
N₂ for flushing of vessels and solution
Gloves in plastic panel & telepliers

### Critical Aspects:

Grade C conditions at rest and during operations
IQ/OQ provided by the equipment supplier
Qualified (sterility, leachables, integrity) disposables by equipment supplier
Process qualification (PQ grade & process) is done

### Grade C/B Material Entry, VHP

### Description of Activity:

Disposable filling materials (provided by machine supplier) and ready to use sterile components (vials, stopper/caps) are added via open door (typically 150 vials per batch)
Items are put on shelves/hung
Box is closed
Secondary packaging is removed via gloves and evacuated
Tubs are placed in transportation unit (“Train”)
EM sampling (start)
VHP Cycle
EM sampling (end)

### Equipment:

Isolatorbox grade C before VHP/Grade B after VHP
Gloves (for removal of primary bag & position into racks)
Glove integrity tester
VHP generator & detection systems & safety warning systems (technical area & clean room) for operator
Hangers/shelves/transportation system

### Critical Aspects:

Grade C/B conditions at rest and during operations
IQ/OQ provided by the equipment supplier
Qualified disposables (sterility, leachables, integrity)
Cycle development (6 log reduction & aeration <1 ppm must be completed within about 120 minutes)
PQ (grade and VHP cycle) is performed by OPERATOR

### VHP Generator(s)

### Description of Activity:

See relevant sections
Each generator should be connectable to
   Material entry unit (sanitized at each material enty)
   Material unpack unit (sanitized per campaign (e.g. daily))
   Filling line unit (sanitized per campaign (e.g. daily))

### Equipment:

generator
Steris or supplier design

### Critical Aspects:

See relevant sections

### A=>Grade A Component Unpack, VHP

### Description of Activity: FASE 1: Preparation Part A

VHP Cycle of empty box (at start of campaign only)
Transfer of material in from material entry box
Components: removal of tyvec layer from tubes; removal of packaging materials from stopper/caps and tubing; optional: automated step
Positioning of components for automatic

### Processing in Filling Unit

Remove waste
EM sampling (during)

### Equipment:

Isolatorbox grade C before VHP/Grade A after VHP
VHP generator & detection systems & safety warning systems (technical area & clean room) for operator
Transportation systems for components
Gloves in lightly shielded panel with glass lead
Glove integrity tester

### Critical Aspects:

Grade C conditions before cycle, Grade A condition after cycle
IQ/OQ provided by the equipment supplier
Cycle development should be done by the equipment supplier (6 log reduction & aeration <1 ppm must be completed within about 120 minutes)
PQ (grade, media fill and VHP) is performed by OPERATOR

### Description of Activity: FASE 1: Operation Part A

No interventions should be needed (except unblock jams): components enter automatically
EM sampling (during)

### Equipment:

See above

### Critical Aspects:

See above

### Description of Activity: FASE 1: End of Operation Part A

End campaign (cleaning) or go back to “preparation A”

### Equipment:

See above

### Critical Aspects:

See above

### B=>Grade A Filling, VHP

### Description of Activity: FASE 1: Preparation Part B

VHP Cycle of empty box (at start of campaign only)
Transfer of material in from previous box
Installation of unpacked disposable components (tubing, needle, . . . )
Connection of tubings/needle/filter to feeding line, pump & needle holder
install N₂ flushing needle
EM sampling (during)
Filter integrity testing (at the start; Wetting with product)
Note: the remainder of the bulk solution stays in the compounding unit

### Equipment:

Isolatorbox grade C before VHP/Grade A after VHP
VHP generator & detection systems & safety warning systems (technical area & clean room) for operator
Gloves in plexipanel & access ports in shielded door & for interventions telepliers/robotic arm-manipulator
Glove integrity tester

### Critical Aspects:

Grade C conditions before cycle
Grade A condition after cycle
IQ/OQ provided by the equipment supplier
Cycle development should be done by the equipment supplier (6 log reduction & aeration <1 ppm must be completed in about 60 to 120 minutes)
PQ (grade, filter integrity test parameters, media fill and VHP) is performed by OPERATOR

### Description of Activity: FASE 1: Operation Part B

Filling, stopper/cap of vial
Reading of 2D code and linkt with filling parameters
No interventions should be needed (except unblock jams)
Product specific: flush with N₂
Optionally fill full area with N₂
EM sampling (during)

### Equipment:

See above (interventions only with telepliers)
Filling & Stoppering/capping unit with integrated balance. Avoidance of post fill dripping is critical.
Camera (reading of 2D code)
Oxygen detection systems & safety warning system for operator
LAF protecting the exit (mousehole: where needed additional valves/shuts need to be installed)

### Critical Aspects:

See above
PQ (see above, filling and closing accuracy) is performed by OPERATOR

### Description of Activity: FASE 1: End of Operations Part B

Run the line empty
Return to normal air (in case of N₂ optional)
Disconnect the feeding line
Remove disposable components to the radioactive waste box
End campaign (cleaning) or go back to “preparation B”
Filter integrity testing

### Equipment:

Filter integrity tester

### Critical Aspects:

See above

### CCIT Inspection, Hold (Prelabeled Lead Pot) for Reject

### Description of Activity:

Vials are presented to CCIT tester
Unit is transferred from CCIT tester
Suspected units are put in a pre-labelled lead pot “suspected CCIT” pending investigation (buffer of 10 vials)
Document and return integer vials back to the line

### Equipment:

CCIT test equipment to be proposed by vendor:
   VD (vacuum decay)
   ME (mass extraction)
Detection of 10 um leaks
Pick and place unit for vial transport
Camera (reading of 2D code)(check on uniqueness at least within batch)
Gloves in plexipanel & access ports in shielded door
Pre-labelled lead pots
Ensure stable temperature/avoid condensation after filling step

### Critical Aspects:

Qualification of testing equipment
IQ/OQ must be provide by the equipment supplier
PQ (equipment validation) is done by OPERATOR

### Dose-Measurement

### Description of Activity:

Vials are presented to dose-calibrator
Feedback is given to the software (see below) and operator if the result is:
   non-conform with the target (x %) or
   deviating from the expectations (bulk measurement & volume) (y %)
   (parameters x and y can be entered in the software)
Unit is transferred (independently of the status)

### Equipment:

Dosecalibrator for finished product
Non conform vials are identified for rejection
Camera (reading of 2D code)
Gloves in plexipanel & access ports in shielded door
Pick and place unit for vial transport

### Critical Aspects:

IQ/OQ must be provide by the equipment supplier
PV (Qualification of process and testing equipment) is done by OPERATOR

### Visual Inspection (VI)

### Description of Activity:

Vials are presented by the machine against black and white background
Rotation speed and light intensity is adjustable
Sequence is video-taped
Sequence is evaluated on screen by production and AQL by quality
Unit is transferred (independently of the visual inspection status)
Any investigation is performed off line

### Equipment:

Camera for visual inspection
Camera for reading of 2D code
Computer screens (remote) and pass/fail keys (production operator(s) & quality)
Data Storage server & software to present sequence to 1 or 2 operators (based on availability) and AQL sample to a quality operator
(see also slide on visual inspection software)
Gloves in plexipanel & access ports in shielded door

### Critical Aspects:

Inspection conditions
   in line with pharmacopea
   (light/duration)
Software qualification
IQ/OQ must be provided by the equipment supplier
PV (process) and operator is done by OPERATOR

### (Optional) Head Space Analysis

### Description of Activity:

Vials are presented to HSA tester
Unit is transferred from HSA tester

### Equipment:

HSA tester (Specifications to be defined)
Pick and place unit for vial transport
Camera (reading of 2D code)
Gloves in plexipanel & access ports in shielded door

### Critical Aspects:

Qualification of testing equipment
IQ/OQ must be provide by the equipment supplier
PQ (equipment validation) is done by OPERATOR

### Labelling Vial & Lead Pot+Seal

### Description of Activity:

Vials are identified by camera for 2D code
Note: the system has to ensure that the correct version of the labels is used (country & revision number)
Paper label is printed and attached to the vial (reject vials are labelled as such*)
Vial is put into a lead container
Paper label is printed and attached to the lead container. Container is sealed. (reject lead pots are labelled as such*)
OPERATOR will evaluate to standardize on a fixed window location for lot-variable information and label ID code. However, variability on color preprinted info is huge, probably require ring in line color printing.
Note: * vials rejected in visual inspection might need relabeling afterwards

### Equipment:

Camera's
   reading of 2D code on vial
   version of preprinted info (country & revision=label ID)
   verifying lot variable information
   Application of seal
   Picture of vial and lead pot labels as part of
      the batch documentation
2 label printers & applicators
Gloves in plexipanel & access ports in shielded door

### Critical Aspects:

Qualification of labelling process
IQ/OQ must be provide by the equipment supplier
PQ is performed by OPERATOR

### Hold (Prelabeled Lead Pot) for Reject; Finished Product; VI; Reject

### Description of Activity:

Lead pots are sorted automatically (fill volume, CCIT, dose, visual inspection, head space (optional) on the release or reject lane.
Up to 150 processed lead pots are collected outside the line.
In addition, units that failed visual inspection are manually sorted out, scanned and added to the “reject” vial units. They are labeled as rejects and receive an extra label (confirmed by scanning)

### Equipment:

Camera to read the label on lead pot
Transportation belt

### Critical Aspects:

Qualification of sorting process
IQ/OQ must be provide by the equipment supplier
PQ is performed by OPERATOR

### Temperature, Relative Humidity, Pressure and Particle Count Monitoring System

The general environmental monitoring system has been installed and validated to ensure the following conditions are properly monitored:
Temperature: 16 to 25° C. (61° F. to 77° F.).
Relative humidity: 28 to 70% RH.
Cooling capacity
Differential pressure
Non-viable particle counts

Particle Counters are used for monitoring the Class A distribution and preparation isolators during production.

In each pharmaceutical area, the pressure differentials are monitored by Digihelic pressure monitoring device.

### Gas Supply System: Compressed Air and Nitrogen

The compressed air used for inflating production/isolator line gaskets is produced from an Atlas SF8+ and an Atlas SF15+ compressed air generators which collect compressed air in a vertical air receiver located in the Air Compressor Room. The compressed air leads to the production lines, the sterility testing laboratories, QC labs, and the Technical Operations Room, passing through PTFE (P)-SRF filters.

Nitrogen is supplied to part of the facility from a compressed nitrogen gas cylinder connected to a distribution manifold in the packaging room. The manifold supplies nitrogen to production lines 1-4, and the QC labs, passing through PTFE (P)-SRF filters. The manifold has the capacity to accommodate up to four cylinders regulated by the manifold itself. An additional single cylinder supplies nitrogen to production lines 5-6 and the Technical Operations Room, passing through PTFE (P)-SRF filters. The distribution lines are built of stainless steel and distribute the gases through the opening/closing of valves.

1. A method for producing a packaged radiopharmaceutical drug product (e.g. a radioligand drug product) comprising the steps of:
(1) Synthesizing and compounding a radiopharmaceutical drug substance solution in one or more shielded units of (cleanliness zoning requirement) Grade C to obtain a radiopharmaceutical drug product;
(2) Providing primary packaging materials, e.g. by (outer-layer-)unpacking in one or more cold units of Grade C, sanitizing in one or more cold units of Grade B, and (inner-layer-)unpacking said packaging materials in one or more cold units of Grade A;
(3) Filling the radiopharmaceutical drug substance solution obtained by step (1) into the primary packaging materials as provided and unpacked by step (2) in a shielded unit of Grade A to obtain the primarily packaged radiopharmaceutical drug product;
(4) Inspecting the primarily packaged radiopharmaceutical drug product obtained by step (3) in one or more shielded units of Grade “Controlled, non-classified” (CNC); and
(5) optionally, placing the primarily packaged radiopharmaceutical drug product as inspected by step (4) into a shielded secondary packaging material in one or more shielded units of Grade CNC to obtained a primarily and secondarily packaged radiopharmaceutical drug product.

2. The method for producing a packaged radiopharmaceutical drug product according to claim 1, wherein in step (4) a container closure integrity test (CCIT) of the primarily packaged radiopharmaceutical drug product is performed as one of the first inspecting steps, preferably, the CCIT is performed with the primarily packaged radiopharmaceutical drug product present in a(n additional) container that allows capturing/segregation of the drug product in said container in case of any leakage detection or risk of spillage.

3. The method for producing a packaged radiopharmaceutical drug product according to claim 1, wherein in step (4) a camera assisted visual inspection (VI) is performed in the form of video recordation, preferably, said video recordation is then evaluated by naked-eye.

4. The method for producing a packaged radiopharmaceutical drug product according to claim 1, wherein in step (1) the synthesizing step is performed several times, preferable up to 4 times, preferably said synthesizing step is performed in parallel in several synthesizing units, and the resulting synthesized radiopharmaceutical drug substance solutions are then collected as one combined (bulk) volume for compounding.

5. The method for producing a packaged radiopharmaceutical drug product according to claim 1, wherein in step (1) the synthesizing step is performed in a cassette-based synthesis units, preferably said cassette-system manufacturing synthesizer unit as provided e.g. by Trasis, preferably MiniAlO as described e.g. in WO 2020/089379 A1, preferably, two double synthesis units by Trasis are used.

6. The method for producing a packaged radiopharmaceutical drug product according to claim 1, wherein in step (1) the compounding step comprises the sub-steps of:
(i) Filling the synthesized solution(s) into a container, e.g. a (bulk) vial or bottle to obtain a (concentrated) mother solution;
(ii) optionally, measuring the radioactivity of said (concentrated) mother solution;
(iii) optionally, transferring said (concentrated) mother solution into another container, preferably a flexible plastic bag,
(iv) adding a dilution solution in an amount to obtain the drug substance solution in the desired radioactive concentration; and
(v) mixing the solution as result of step (iv), preferably and if applicable, by moving the flexible plastic bag appropriately, to obtain a homogeneous diluted drug substance solution for filling.

7. The method for producing a packaged radiopharmaceutical drug product according to claim 1, wherein in step (2) the packaged primary packaging material is (bio)decontaminated/sanitized, preferably with vaporized hydrogen peroxide (VHP), before being transferred from the unit of Grade B to the unit of Grade A.

8. The method for producing a packaged radiopharmaceutical drug product according to claim 1, wherein in step (4) inspecting comprises the following sub-steps:
(i) CCIT;
(ii) radioactive dose measurement/calibration;
(iii) visual inspection; and
(iv) optionally, head space analysis;
preferably in said order.

9. The method for producing a packaged radiopharmaceutical drug product according to claim 1, wherein the primary packaging material comprises a glass vial and an all-plastic push-fit cap, wherein the rubber stopper is pre-assembled in the cap, for example, a RayDyLyo cap by ARaymond.

10. The method for producing a packaged radiopharmaceutical drug product according to claim 1, wherein the shielded secondary packaging material comprises a lead pot with a corresponding lid, preferably with a tamper evident application, preferably said lead pot and its lid is free of a paint-coating, preferably said lead pot and its lid are plastic-coated, preferably a rubber ring is present between pot and lid for leakage-safe closure.

11. The method for producing a packaged radiopharmaceutical drug product according to claim 1, wherein the drug substance carrying containers and tubings connecting said containers are flushed with protective gas/inert gas, for example nitrogen (N₂) and/or argon (Ar).

12. The method for producing a packaged radiopharmaceutical drug product according to claim 1, wherein the solutions involved in the method are transferred from one container to the other via tubings by applying overpressure of protective gas/inert gas, for example nitrogen (N₂) and/or argon (Ar), preferably, said transfer is preceded by testing the tubings for flow and leakage, e.g. by a pressure test with said protective gas/inert gas.

13. The method for producing a packaged radiopharmaceutical drug product according to claim 1, wherein the solutions used in the method are purged and/or flushed with protective gas/inert gas, for example nitrogen (N₂) and/or argon (Ar).

14. The method for producing a packaged radiopharmaceutical drug product according to claim 1, wherein the at least the steps (1)-(3) are automated, preferably also the steps (4) and (5) are automated, with the exception of the naked-eye evaluation of the video recordation in step (4).

15. The method for producing a packaged radiopharmaceutical drug product according to claim 1, wherein said product is produced on a scale of at least 30 Ci, preferably at least 32 Ci, more preferably 35 Ci, even more preferably at least 40 Ci.

16. A packaged radiopharmaceutical drug product obtained (and/or obtainable) by the method of claim 1.

17. A manufacturing unit (e.g. in the form of a(n automated) process line) comprising the technical equipment and facilities for producing a packaged radiopharmaceutical drug product according to the method of claim 1.

18. Use of the manufacturing unit according to claim 17 for producing the packaged radiopharmaceutical drug product.

19. A method of treating a cancer comprising administering to a patient in need thereof a therapeutically effective amount of the radiopharmaceutical drug product contained in the packaged radiopharmaceutical drug product according to claim 16.

20. The radiopharmaceutical drug product contained in the packaged radiopharmaceutical drug product according to claim 16 for use in the treatment of a cancer.

21. Use of the radiopharmaceutical drug product contained in the packaged radiopharmaceutical drug product according to claim 16 for the preparation of a medicament for the treatment of a cancer.
